(19) United States
(12) Patent Application Publication
(10) Pub. No.: US 2024/0011981 A1
(43) Pub. Date: Jan. 11, 2024

(54) Two-Dimensional Multiplex Chromatography Test Card

(71) Applicant: Bioteke Corporation (Wuxi) Co., Ltd., Wuxi (CN)

(72) Inventor: Zhitu ZHOU, Wuxi (CN)

(73) Assignee: Bioteke Corporation (Wuxi) Co., Ltd., Wuxi (CN)

(21) Appl. No.: 17/893,159

(22) Filed: Aug. 22, 2022

(30) Foreign Application Priority Data
Jul. 5, 2022 (CN) 202221720626.6

(51) Int. Cl. G01N 33/543; G01N 33/569; G01N 33/577; C07K 16/10; G01N 33/58

(52) U.S. Cl. CPC G01N 33/54389; G01N 33/56983; G01N 33/577; C07K 16/1027; G01N 33/585

(57) ABSTRACT

A two-dimensional multiplex chromatography test card is disclosed. The two-dimensional multiplex chromatography test card comprises a first absorbent pad (11), a first nitrocellulose membrane (10), a sample pad (1), a second nitrocellulose membrane (3) and a second absorbent pad (4) which are sequentially provided on a backing (5) in a lapping manner along a length direction of the backing (5). A first conjugating zone (9) on the sample pad (1) is coated with at least one first latex bead labeled antibody, and a second conjugating zone (2) on the sample pad (1) is coated with at least one second latex bead labeled antibody. The first nitrocellulose membrane (10) is provided with a first test line (12 and/or 13) and a first control line (14), and the first test line (12 and/or 13) is coated with a first antibody corresponding to the first latex bead labeled antibody. The second nitrocellulose membrane (3) is provided with a second test line (6 and/or 7) and a second control line (8).

### CROSS REFERENCE TO RELATED APPLICATION

This patent application claims the benefit and priority of Chinese Patent Application No. 202221720626.6, filed on Jul. 5, 2022, the disclosure of which is incorporated by reference herein in its entirety as part of the present application.

### TECHNICAL FIELD

The present disclosure relates to the field of bioassays, and in particular relates to a two-dimensional multiplex chromatography test card.

### BACKGROUND ART

Immunochromatography is that a specific antibody is immobilized in a certain zone of a nitrocellulose membrane, when one end of the dried nitrocellulose membrane is infiltrated into a sample, the sample will move forward along the membrane due to the capillary action, and when moving to the region where the antibody is immobilized, the corresponding antigen in the sample is specifically conjugated to the antibody, and if it is labeled and stained with immunocolloidal gold or other immunology markers, the region may show a certain color, thereby achieving specific immunodiagnosis. At present, the testing of a plurality of to-be-tested indexes usually requires a plurality of test cards corresponding to the various indexes to complete, which increases the number of user operations and makes the convenience of testing low. The production of each test card needs to cut and paste the sample pad, the conjugate pad, the nitrocellulose membrane and the absorbent pad, resulting in low production efficiency.

### SUMMARY

The present disclosure provides a two-dimensional multiplex chromatography test card, aiming at solve the technical problems of low convenience for the testing of a plurality of to-be-tested indexes and low production efficiency of test cards in the prior art.

To achieve the objective above, the present disclosure provides a two-dimensional multiplex chromatography test card. The two-dimensional multiplex chromatography test card comprises a first absorbent pad, a first nitrocellulose membrane, a sample pad, a second nitrocellulose membrane and a second absorbent pad which are sequentially provided on a backing along a length direction of the backing.

Both ends of the sample pad are respectively lapped to one end of the first nitrocellulose membrane and one end of the second nitrocellulose membrane; The end, away from the sample pad, of the first nitrocellulose membrane is lapped to the first absorbent pad, and the end, away from the sample pad, of the second nitrocellulose membrane is lapped to the second absorbent pad.

A first conjugating zone on the sample pad is coated with at least one first latex bead labeled antibody, and a second conjugating zone on the sample pad is coated with at least one second latex bead labeled antibody.

The first nitrocellulose membrane is provided with at least one first test line and a first control line, and each of the first test lines is coated with a first antibody corresponding to the first latex bead labeled antibody.

The second nitrocellulose membrane is provided with at least one second test line and a second control line, and each of the second test lines is coated with a second antibody corresponding to the second latex bead labeled antibody.

Alternatively, latex beads in each of the first latex bead-labeled antibodies and each of the second latex bead-labeled antibodies have the same or different colors.

Alternatively, the first latex bead labeled antibody comprises an influenza A labeled antibody labeled with latex beads, an influenza B labeled antibody labeled with latex beads, or a novel coronavirus labeled antibody labeled with latex beads; and the second latex bead-labeled antibody comprises an influenza A labeled antibody labeled with latex beads, an influenza B labeled antibody labeled with latex beads, or a novel coronavirus labeled antibody labeled with latex beads.

Alternatively, the control line is coated with a goat anti-mouse IgG antibody.

Alternatively, the sample pad is obtained by infiltrating the sample pad in sample pad treatment buffer and then drying, wherein the sample pad treatment buffer comprises Tris(hydroxymethyl)aminomethane, casein, and Tween-20.

Alternatively, the first conjugate pad is 3 mm to 5 mm away from an edge of one end of the sample pad, and the second conjugate pad is 3 mm to 5 mm away from an edge of one end of the sample pad.

Alternatively, the lapping part has a lapping length of 1 to 3 mm.

Alternatively, the coating amount of each of the first antibodies is 0.1 to 2.0 mg/mL and the coating amount of each of the second antibodies is 0.1 to 2.0 mg/mL.

Alternatively, the concentration of the latex beads in each of the first latex bead labeled antibodies is 0.2% to 0.4%, and the concentration of the latex beads in each of the second latex bead labeled antibodies is 0.2% to 0.4%.

Alternatively, the latex beads in the first latex bead-labeled antibody and in the second latex bead-labeled antibody have a particle size of 300 to 500 nm.

The present disclosure provides a two-dimensional multiplex chromatography test card. A first absorbent pad, a first nitrocellulose membrane, a sample pad, a second nitrocellulose membrane and a second absorbent pad are sequentially provided on a backing. Both ends of the sample pad are respectively lapped to one end of the first nitrocellulose membrane and one end of the second nitrocellulose membrane; The end, away from the sample pad, of the first nitrocellulose membrane is lapped to the first absorbent pad, and the end, away from the sample pad, of the second nitrocellulose membrane is lapped to the second absorbent pad, thereby forming a test structure for two-dimensional chromatography from the sample pad, serving as the center, to both sides, such that two or more indexes can be tested by one-time sample injection when a user performs the multi-index test, sample injection times during testing is reduced, and the testing operation is simplified. A first conjugating zone on the sample pad is coated with at least one first latex bead labeled antibody, and a second conjugating zone on the sample pad is coated with at least one second latex bead labeled antibody. The first nitrocellulose membrane is provided with at least one first test line and a first control line, and each of the first test lines is coated with a first antibody corresponding to the first latex bead labeled antibody; the second nitrocellulose membrane is provided with at least one second test line and a second control line, and each of the second test lines is coated with a second antibody corresponding to the second latex bead labeled antibody. Therefore, the two-in-one of the sample pad and the conjugate pad is achieved, the process of cutting the conjugate pad and the process of assembling the sample pad and the conjugate pad by lapping and attaching are reduced, the production efficiency is improved, and the technical problems of low convenience for the testing of a plurality of to-be-tested indexes and low production efficiency of test cards in the prior art are overcome.

### BRIEF DESCRIPTION OF THE DRAWINGS

The present disclosure will be described with regard to the accompanying drawings, which assist in illustrating various features of the disclosure. It should be appreciated that the attached figures in the following description are provided solely for the purpose of illustrating the practice of the disclosure and do not constitute limitations on the scope thereof as certain variations and modifications, other variations and modifications are well within the skill and knowledge of one of ordinary skill after having read the present disclosure.

FIG. 1 is a structure diagram of an embodiment I of a two-dimensional multiplex chromatography test card in accordance with the present disclosure.

In the drawings:

| Reference | | Reference | |
|---|---|---|---|
| number | Name | number | Name |
| 1 | Sample pad | 2 | Second conjugating zone |
| 3 | Second nitrocellulose | 4 | Second absorbent pad |
| | membrane | | |
| 5 | Backing | 6 | Second test line away |
| | | | from sample pad |
| 7 | Second test line close | 8 | Second control line |
| | to sample pad | | |
| 9 | First conjugating zone | 10 | First nitrocellulose |
| | | | membrane |
| 11 | First absorbent pad | 12 | First test line away |
| | | | from sample pad |
| 13 | First test line close to | 14 | First control line |
| | sample pad | | |

### DETAILED DESCRIPTION OF THE EMBODIMENTS

It should be noted that if there is a directional indication (such as up, down, left, right, front, back . . . ) in the embodiments of the present disclosure, the directional indication is used only to explain a relative positional relationship between components, motion situations, etc. at a certain specific attitude (as shown in the figures). If the specific attitude changes, the directional indication also correspondingly changes.

In addition, if there are descriptions related to “first”, “second”, etc. in the embodiments of the present disclosure, the descriptions of “first”, “second”, etc. are used for descriptive purposes only and are not to be construed as indicating or implying relative importance or implying a number of the indicated technical features. As such, the features limited to “first” and “second” may explicitly or implicitly comprise at least one of those features. In addition, if “and/or” appears in the full text, it comprises three parallel solutions, “A and/or B” is taken as an example, including solution A, solution B, or solutions that satisfy both A and B.

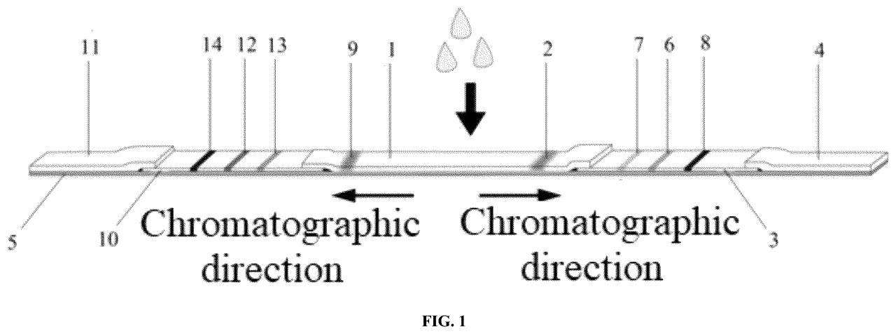

The present disclosure provides a two-dimensional multiplex chromatography test card. In accordance with the embodiment of the present disclosure, refer to FIG. 1, the two-dimensional multiplex chromatography test card comprises a first absorbent pad (11), a first nitrocellulose membrane (10), a sample pad (1), a second nitrocellulose membrane (3) and a second absorbent pad (4) which are sequentially lapped to a backing (5) in a length direction of the backing (5).

Both ends of the sample pad (1) are respectively lapped to one end of the first nitrocellulose membrane (10) and one end of the second nitrocellulose membrane (3); the end, away from the sample pad (1), of the first nitrocellulose membrane (10) is lapped to the first absorbent pad (11), and the end, away from the sample pad (1), of the second nitrocellulose membrane (3) is lapped to the second absorbent pad (4).

A first conjugating zone (9) on the sample pad (1) is coated with at least one first latex bead labeled antibody, and a second conjugating zone (2) on the sample pad 1 is coated with at least one second latex bead labeled antibody.

The first nitrocellulose membrane (10) is provided with at least one a first test line and a first control line (14), and each of the first test lines is coated with a first antibody corresponding to the first latex bead labeled antibody.

The second nitrocellulose membrane (3) is provided with at least one second test line and a second control line (8), and each of the second test lines is coated with a second antibody corresponding to the second latex bead labeled antibody.

Specifically, the two-dimensional multiplex chromatography test card comprises a backing (5) which is rectangular. A first absorbent pad (11), a first nitrocellulose membrane (10), a sample pad (1), a second nitrocellulose membrane (3) and a second absorbent pad (4) are sequentially arranged on the backing (5) in a lapping manner along a length direction of the backing (5). The sample pad (1) is fixedly arranged at a middle position of the backing (5). Both ends of the sample pad (1) are respectively lapped to the first nitrocellulose membrane (10) and the second nitrocellulose membrane (3). The end, away from the sample pad (1), of the first nitrocellulose membrane (10) is lapped to the first absorbent pad (11), and the end, away from the sample pad (1), of the second nitrocellulose membrane (3) is lapped to the second absorbent pad (4).

The sample pad (1) comprises a first conjugating zone (9) and a second conjugating zone (2) which are respectively located at positions, close to the edges, at both ends of an extending direction of the sample pad (1). The first conjugating zone (9) is coated with at least one first latex bead-labelled antibody, such that each of the first latex bead labeled antibodies coated on the first conjugating zone (9) is redissolved when a liquid sample flows through the first conjugating zone (9). The second conjugating zone (2) is coated with at least one second latex bead labeled antibody, such that each of the second latex bead labeled antibodies coated on the second conjugating zone (2) is redissolved when a liquid sample flows through the second conjugating zone (2). The first latex bead labeled antibody is an antibody labeled with first latex beads, the second latex bead labeled antibody is an antibody labeled with second latex beads, and each of the first latex bead labeled antibodies may be the same as or different from each of the second latex bead labeled antibodies. In a mode of spraying and coating the latex bead labeled antibodies on the sample pad, the conjugate pad and the sample pad (1) are conjugated, that is, there is no separate conjugate pad in the two-dimensional multiplex chromatography test card of the present disclosure, and there is no need to prepare the conjugate pad separately, such that the process of assembling and attaching the conjugate pad to the test card is reduced, the production process is simplified, and the production efficiency is improved.

The first nitrocellulose membrane (10) is provided with at least one first test line and a first control line (4), each of the first test lines is coated with a first antibody corresponding to the first latex bead labeled antibody. The second nitrocellulose membrane (3) is provided with at least one second test line and a second control line (8), each of the second test lines is coated with a second antibody corresponding to the second latex bead labeled antibody, where each of the first antibodies may be the same as or different from each of the second antibodies.

It needs to be noted that specific types of the latex bead labeled antibody coated on the conjugating zone and the antibodies coated on the corresponding test line of the conjugating zone may be determined according to the index actually needing to be tested. The index may comprise two or more, for example, the index may comprise both of influenza A and novel coronavirus, or may comprise influenza A, influenza B and novel coronavirus, or may comprise novel coronavirus, respiratory syncytial virus, influenza A and influenza B, etc.

Alternatively, latex beads of each of the first latex bead labeled antibodies and each of the second latex bead labeled antibodies have the same or different colors.

In accordance with this embodiment, specifically, different antibodies are labeled with the latex beads having different colors, and then different indexes will be shown by bands having different colors during the final color development. A user may intuitively and accurately read the test results according to the color of the band, especially when only one of a plurality of test results is positive, such that the probability of misjudgment may be reduced, the time spent in determining which index the band corresponds to by comparing the distances can be reduced, and the test efficiency is improved.

Alternatively, the first latex bead labeled antibody comprises an influenza A labeled antibody labeled with latex beads, an influenza B labeled antibody labeled with latex beads, or a novel coronavirus labeled antibody labeled with latex beads. The second latex bead-labeled antibody comprises an influenza A labeled antibody labeled with latex beads, an influenza B labeled antibody labeled with latex beads, or a novel coronavirus labeled antibody labeled with latex beads.

In accordance with this embodiment, specifically, any two indexes in the influenza A virus, the influenza B virus and the novel coronavirus may be tested on one side of the two-dimensional multiplex chromatography test card, and the other index in the influenza A virus, the influenza B virus and the novel coronavirus may be detected on the other side of the two-dimensional multiplex chromatography test card, and thus the testing of the influenza A virus, the influenza B virus and the novel coronavirus may be achieved by using one two-dimensional multiplex chromatography test card, dropping the sample solution one time and performing testing one time. As the influenza A virus, the influenza B virus and the novel coronavirus have similar infection symptoms and susceptibility time, one test card may test three viruses at the same time, and may complete the determination and identification of three antigens one time, and thus has a certain practical significance.

Alternatively, the control line is coated with a goat anti-mouse IgG antibody.

Alternatively, the sample pad (1) is obtained by infiltrating the sample pad in sample pad treatment buffer and then drying, wherein the sample pad (1) treatment buffer comprises Tris(hydroxymethyl)aminomethane, casein, and Tween-20.

In accordance with this embodiment, specifically, the sample pad (1) is obtained by infiltrating the sample pad in the sample pad (1) treatment buffer until completely infiltrated, draining the excess liquid and then drying at 55° C., wherein the sample pad 1 treatment buffer is 0.1 M Tris(hydroxymethyl)aminomethane, 0.1% to 1.0% casein, and 0.1% to 1.0% Tween-20.

Alternatively, the first conjugating zone (9) is 3 mm to 5 mm away from an edge of one end of the sample pad (1), and the second conjugating zone (2) is 3 mm to 5 mm away from an edge of one end of the sample pad (1).

In accordance with this embodiment, specifically, the first conjugating zone (9) is located at one end, close to the first nitrocellulose membrane (10), on the sample pad (1), and is 3 mm to 5 mm away from the edge of one end, close to the first nitrocellulose membrane (10), in the sample pad (1), such as, 3 mm, 4.2 mm, and 5 mm. The second conjugating zone (2) is located at one end, close to the second nitrocellulose membrane (3), on the sample pad (1), and is 3 mm to 5 mm away from the edge of one end, close to the second nitrocellulose membrane (3), in the sample pad (1), such as, 3 mm, 3.8 mm, and 5 mm.

Alternatively, the lapping part has a lapping length of 1 mm to 3 mm.

In accordance with this embodiment, specifically, the lapping length of the lapping part should make the liquid sample flow to the nitrocellulose membrane from the sample pad (1) better. However, the longer the lapping length is, the more the consumed liquid sample is, and therefore, it is determined that the lapping part has a lapping length of 1 mm to 3 mm, such as 1 mm, 2.1 mm, and 3 mm.

Alternatively, the coating amount of each of the first antibodies is 0.1 mg/mL to 2.0 mg/mL and the coating amount of each of the second antibodies is 0.1 mg/mL to 2.0 mg/mL.

In accordance with this embodiment, the coating amount of each of the first antibodies is 0.1 mg/mL to 2.0 mg/mL, such as 0.1 mg/mL, 1.4 mg/mL, 2.0 mg/mL. The coating amount of each of the second antibodies is 0.1 mg/mL to 2.0 mg/mL, such as 0.1 mg/mL, 0.9 mg/mL, 2.0 mg/mL.

Alternatively, the concentration of the latex beads in each of the first latex bead labeled antibodies is 0.2% to 0.4%, and the concentration of the latex beads in each of the second latex bead labeled antibodies is 0.2% to 0.4%.

In accordance with this embodiment, the concentration of the latex beads in each of the first latex bead labeled antibodies is 0.2% to 0.4%, such as 0.2%, 0.3% and 0.4%. The concentration of the latex beads in each of the second latex bead labeled antibodies is 0.2% to 0.4%, such as 0.2%, 0.3% and 0.4%. The labeled antibodies may be directly sprayed onto the sample pad (1) by means of a spraying technology. By optimizing the concentration of the labeled antibody, the labeled antibody may be directly sprayed onto the sample pad (1), such that the steps of cutting and fixing when pasting the sheet may be eliminated, and the production process is simplified.

Alternatively, the latex beads in each of the first latex bead labeled antibodies and in each of the second latex bead labeled antibodies have a particle size of 300 nm to 500 nm.

In accordance with this embodiment, specifically, the particle sizes of the latex beads in the first latex bead labeled antibody and the second latex bead labeled antibody are in the range of 300 nm to 500 nm, which are larger than the particle size of 20 to 60 nm of the conventional colloidal gold, and thus the sensitivity is improved. During practical testing, the sensitivity is improved by about 2 to 10 times. Due to the fact that the beads having different colors are used in different items, the problems can be better found in the early research and development debugging, thus avoiding false positive from the source, and improving the quality of the product.

In an implementable manner, when the two-dimensional multiple chromatography test card provided by this embodiment is used for testing, referring to FIG. 1, the second conjugating zone of the two-dimensional multiple chromatography test card is coated with an A-item labeled antibody labeled with blue latex beads and a B-item labeled antibody labeled with red latex beads. The first conjugating zone (9) of the two-dimensional multiplex chromatography test card is coated with C-item labeled antibody labeled with green latex beads and D-item labeled antibody labeled with black latex beads. A second test line (7) close to the sample pad is coated with an A-item coated antibody, A second test line (6) away from the sample pad is coated with a B-item coated antibody, a first test line (13) close to the sample pad is coated with a C-item coated antibody, and a first test line (12) away from the sample pad is coated with a D-item coated antibody.

The specific preparation process of the two-dimensional multiplex chromatography test card comprises the following steps:

(1) Preparation of Nitrocellulose Membrane for Multiplex Testing:

Respectively diluting commercial goat anti-mouse IgG antibody, an A test item dispensing antibody, a B test item dispensing antibody, a C test item dispensing antibody and a D test item dispensing antibody to 0.1 mg/mL to 2.0 mg/mL, 0.1 mg/mL to 2.0 mg/mL, 0.1 mg/mL to 2.0 mg/mL, 0.1 mg/mL to 2.0 mg/mL and 0.1 mg/mL to 2.0 mg/mL with coating buffer; adjusting a position of a dispensing tip according to a prepared dispensing template, and dispensing, with a spray volume of 1 μL/cm, in sequence at the corresponding position, and then placing the dispensed nitrocellulose membrane into an oven at 55° C. to be baked for 2 hours, wherein the coating buffer comprises: 20 mM of PBS(phosphate buffered saline) and 5% of trehalose.

(2) Preparation of an A-Item Labeled Antibody Labeled with Blue Latex Beads:

Diluting beads to 1% with an MES (Morpholinoethanesulfonic Acid) buffer with pH of 6.5, adding EDC(dichloroethane) which is 0.05 times the mass of the beads, uniformly mixing, then adding 0.1 mg/mL to 2.0 mg/mL A-item antibody, rotating and uniformly mixing at room temperature and leaving overnight; centrifuging to remove supernatant, adding the same volume of a labeling blocking buffer as the MES, uniformly mixing ultrasonically, and rotating and blocking for 1 h; centrifuging to remove supernatant, re-dissolving with the labeling blocking buffer and storing, and placing at 4° C. for further use. Wherein the labeling blocking buffer comprises 0.1 M of Tris (Tris(hydroxymethyl)aminomethane), and a mixture of one or more of 0.1-1.0% of fish skin gelatin, BSA (Bovine albumin), and Casein (casein).

(3) Preparation of a B-Item Labeled Antibody Labeled with Red Latex Beads:

Diluting beads to 1% with an MES buffer with pH of 6.5, adding EDC which is 0.05 times the mass of the beads, uniformly mixing, then adding 0.1 mg/mL to 2.0 mg/mL B-item antibody, rotating and uniformly mixing at room temperature and leaving overnight; centrifuging to remove supernatant, adding the same volume of a labeling blocking buffer as the MES, uniformly mixing ultrasonically, and rotating and blocking for 1 h; centrifuging to remove supernatant, re-dissolving with the labeling blocking buffer, and storing, and placing at 4° C. for further use. Wherein the labeling blocking buffer is 0.1 M of Tris, and a mixture of one or more of 0.1% to 1.0% of fish skin gelatin or BSA or Casein.

(4) Preparation of a C-Item Labeled Antibody Labeled with Green Latex Beads:

Diluting beads to 1% with an MES buffer with pH of 6.5, adding EDC which is 0.05 times the mass of the beads, uniformly mixing, then adding 0.1 mg/mL to 2.0 mg/mL C-item antibody, rotating and uniformly mixing at room temperature and leaving overnight; centrifuging to remove supernatant, adding the same volume of a labeling blocking buffer as the MES, uniformly mixing ultrasonically, and rotating and blocking for 1 h; centrifuging to remove supernatant, re-dissolving with the labeling blocking buffer, and storing, and placing at 4° C. for further use. Wherein the labeling blocking buffer is 0.1 M of Tris, and a mixture of one or more of 0.1% to 1.0% of fish skin gelatin or BSA or Casein.

(5) Preparation of a D-Item Labeled Antibody Labeled with Black Latex Beads:

Diluting beads to 1% with an MES buffer with pH of 6.5, adding EDC which is 0.05 times the mass of the beads, uniformly mixing, then adding 0.1 mg/mL to 2.0 mg/mL D-item antibody, rotating and uniformly mixing at room temperature and leaving overnight; centrifuging to remove supernatant, adding the same volume of a labeling blocking buffer as the MES, uniformly mixing ultrasonically, and rotating and blocking for 1 h; centrifuging to remove supernatant, re-dissolving with the labeling blocking buffer, and storing, and placing at 4° C. for further use. Wherein the labeled blocking buffer is 0.1 M Tris, and a mixture of one or more of 0.1% to 1.0% fish skin gelatin or BSA or Casein.

(6) Preparation of a Sample Pad (1):

Treating the sample pad (1) with a sample pad treatment buffer, infiltrating the sample pad until the sample pad is completely infiltrated, draining recess buffer, drying at 55° C. for further use, wherein the sample pad treatment buffer is 0.1M of Tris, 0.1-1.0% of Casein, and 0.1-1.0% of Tween-20 (Tween-20).

(7) Preparation of a Second Conjugating Zone (2):

Respectively diluting the concentration of the latex beads in the A-item labeled antibody and the B-item labeled antibody to a final concentration of 0.3% with a bead diluent, then spraying the solution to the sample pad (1) by using a machine, with a spray volume of 2.0 μL/cm, and placing the sprayed sample pad (1) at 55° C. to be baked for 30 min; and then taking out the sample pad for further use. Wherein the bead diluent is one or more of 0.1 M of Tris, 0.1% to 1.0% of Casein, 0.1% to 1.0% of polyvinyl pyrrolidone (PVP), 1 to 5% of saccharose, and mycose.

(8) Preparation of a First Conjugate Pad:

Respectively diluting the concentration of the latex beads in the C-item labeled antibody and the D-item labeled antibody to a final concentration of 0.3% with a bead diluent, then spraying the solution to a sample by using a machine, with a spray volume of 2.0 μL/cm, and placing the sprayed conjugate pad at 55° C. to be baked for 30 min; and taking out the conjugate pad for further use. wherein the bead diluent is one or more of 0.1 M of Tris, 0.1% to 1.0% of Casein, 0.1% to 1.0% of (PVP), 1% to 5% of saccharose, and mycose.

(9) Assembling of Test Card:

Attaching absorbent paper to both ends of a length direction of a backing (5) to which two nitrocellulose membranes have been attached so as to cover the nitrocellulose membranes for about 1 mm, wherein the other ends of the nitrocellulose membrane are covered with the sample pad (1) coated with the latex beads after directions are adjusted well, and both ends of the sample pad are covered with the nitrocellulose membrane for about 1 mm; and cutting the backing into strips for further use.

During practical test, the treated sample is dropped to the middle zone of the sample pad (1), and the sample naturally faints on the sample pad (1), and then chromatography is carried out to the left and right sides under the capillary action.

The liquid at the right side firstly flows through the second conjugating zone (2) to redissolve the A-item labeled antibody labeled with blue latex beads and the B-item labeled antibody labeled with red latex beads which are coated on the second conjugating zone (2), and then the chromatography is continuously carried out towards the right side. When there is a certain amount of A-item antigen (i.e., the A item is positive) in the sample, a complex of A-item antigen-A-item labeled antibody-blue latex beads is formed for continuous chromatography rightwards. When reaching the second test line (7) close to the sample pad, the complex is captured by the A-item coated antibody which is coated and immobilized in advance by the second test line (7) close to the sample pad to form a complex of A-item coated antibody-A-item antigen-A-item labeled antibody-blue latex beads, with a blue line visible to naked eyes. Similarly, when there is a certain amount of B-item antigen (i.e., the B item is positive) in the sample, a complex of the B-item antigen-B-item labeled antibody-red latex beads is formed for continuous chromatography rightwards. When reaching a second test line (6) close to the sample pad, the complex is captured by the B-item coated antibody which is coated and immobilized in advance by the second test line (6) close to the sample pad to form a complex of B-item coated antibody-B-item antigen-B-item labeled antibody-red latex beads, with a red line visible to naked eyes. The excess unconjugated A-item antibody labeled with blue latex beads and B-item antibody labeled with red latex beads are continuously subjected to chromatography rightwards, and are captured by goat anti-mouse IgG antibody coated and immobilized at a second control line (8) so as to form a complex of goat anti-mouse IgG-A-item antibody-blue latex beads and a complex of goat anti-mouse IgG-B-item antibody-red latex beads, with a purple-red line visible to naked eyes.

The liquid at the left side firstly flows through the first conjugating zone (9) to redissolve the C-item labeled antibody labeled with green latex beads and the D-item labeled antibody labeled with black latex beads which are coated on the first conjugating zone (9), and then chromatography is continuously carried out towards the left side. When there is a certain amount of C-item antigen (i.e., the C item is positive) in the sample, a complex of the C-item antigen-C-item labeled antibody-green latex beads is formed for continuous chromatography leftwards. When reaching a first test line (13) close to the sample pad, the complex is captured by the C-item coated antibody which is coated and immobilized in advance by the first test line (13) close to the sample pad to form a complex of C-item coated antibody-C-item antigen-C-item labeled antibody-green latex beads, with a green line visible to naked eyes. Similarly, when there is a certain amount of D-item antigen (i.e., the D item is positive) in the sample, a complex of the D-item antigen-D-item labeled antibody-black latex beads is formed for continuous chromatography leftward. When reaching a first test line (12) close to the sample pad, the complex is captured by the D-item coated antibody which is coated and immobilized in advance by the first test line (12) close to the sample pad to form a complex of D-item coated antibody-D-item antigen-D-item labeled antibody-black latex beads, with a black line visible to naked eyes. The excess unconjugated C-item antibody labeled with green latex beads and D-item antibody labeled with black latex beads are continuously subjected to chromatography leftwards, and are captured by goat anti-mouse IgG antibody coated and immobilized at a first control line (14) so as to form a complex of goat anti-mouse IgG-C-item antibody-green latex beads and a complex of goat anti-mouse IgG-D-item antibody-black latex beads, with a dark green line visible to naked eyes.

When there is no a certain to-be-tested antigen in the sample, the band with the corresponding color will not appear at the corresponding position on the nitrocellulose membrane. However, regardless of the presence or absence of the to-be-tested antigen, the sample will be captured by the goat anti-mouse IgG antibody at the second control line (8) on the right side and the first control line (14) on the left side so as to form control line bands visible to naked eyes. If there is no control line band at either end or both ends of both sides, it will be regarded as invalid and retesting is needed.

In accordance with this embodiment, a first absorbent pad, a first nitrocellulose membrane, a sample pad, a second nitrocellulose membrane and a second absorbent pad are sequentially provided on a backing. Both ends of the sample pad are respectively lapped to one end of the first nitrocellulose membrane and one end of the second nitrocellulose membrane; The end, away from the sample pad, of the first nitrocellulose membrane is lapped to the first absorbent pad, and the end, away from the sample pad, of the second nitrocellulose membrane is lapped to the second absorbent pad, thereby forming a test structure for two-dimensional chromatography from the sample pad, serving as the center, to both sides, such that two or more indexes can be tested by one-time sample injection when a user performs the multi-index test, sample injection times during testing is reduced, and the testing operation is simplified. A first conjugating zone on the sample pad is coated with at least one first latex bead labeled antibody, and a second conjugating zone on the sample pad is coated with at least one second latex bead labeled antibody. The first nitrocellulose membrane is provided with at least one first test line and a first control line, and each of the first test lines is coated with a first antibody corresponding to the first latex bead labeled antibody; the second nitrocellulose membrane is provided with at least one second test line and a second control line, and each of the second test lines is coated with a second antibody corresponding to the second latex bead labeled antibody. Therefore, the two-in-one of the sample pad and the conjugate pad is achieved, the process of cutting the conjugate pad and the process of assembling the sample pad and the conjugate pad by lapping and attaching are reduced, the production efficiency is improved, and the technical problems of low convenience for the testing of a plurality of to-be-tested indexes and low production efficiency of test cards in the prior art are overcome.

The following describes the two-dimensional multiplex chromatography test card of the present disclosure in detail with specific embodiments. It is to be understood that the following description is exemplary only and is not a specific limitation to the present disclosure.

### EMBODIMENT I

A triple test card for novel coronavirus/influenza A/influenza B comprises a first absorbent pad, a first nitrocellulose membrane, a sample pad, a second nitrocellulose membrane and a second absorbent pad which are sequentially provided on a backing, wherein both ends of the sample pad are respectively lapped to one end of the first nitrocellulose membrane and one end of the second nitrocellulose membrane. The end, away from the sample pad, of the first nitrocellulose membrane is lapped to the first absorbent pad, and the end, away from the sample pad, of the second nitrocellulose membrane is lapped to the second absorbent pad.

A first conjugating zone on the first sample pad is coated with a novel coronavirus antibody labeled with latex beads, a second conjugating zone on the sample pad is coated with an influenza A antibody labeled with latex beads and an influenza B antibody labeled with latex beads.

The first nitrocellulose membrane is provided with a first test line and a first control line, the first test line is coated with the coronavirus antibody, and the first control line is coated with a goat anti-mouse IgG antibody.

The second nitrocellulose membrane is provided with two test lines and a second control line, one of the second test lines is coated with the influenza A antibody, the other of the second test lines is coated with the influenza B antibody, and the second control line is coated with the goat anti-mouse IgG antibody.

The embodiment I is compared with a conventional test card available on the market, with comparison results as shown in Table 1:

TABLE 1
Testing results of Embodiment I and Conventional Test card

| Comparative | | | | | | | |
|---|---|---|---|---|---|---|---|
| Item | | Embodiment I: | | | Conventional test card | | |
| Item name | | Novel | Influenza | Influenza | Novel | Influenza | Influenza |
| | | coronavirus | A | B | coronavirus | A | B |
| Production | | Excellent | | | Poor | | |
| efficiency | | | | | | | |
| Sample | | One time (Three drops) | | | Three times (Nine drops) | | |
| injection | | | | | | | |
| times | | | | | | | |
| Color | | Multi-color | | | Single color | | |
| development | | | | | | | |
| effect | | | | | | | |
| Negative | | − | − | − | − | − | − |
| matrix | | | | | | | |
| 10 | pg/ml | + | + | + | ± | ± | ± |
| 20 | pg/ml | ++ | ++ | ++ | + | + | + |
| 40 | pg/ml | ++ | ++ | ++ | ++ | ++ | ++ |
| 80 | pg/ml | +++ | +++ | +++ | +++ | +++ | +++ |
| 160 | pg/ml | +++ | +++ | +++ | +++ | +++ | +++ |

It may be known from the Table 2 that, compared with the conventional test card, the Embodiment I is more productive, less in sample injection times, and more obvious and clearer in color development, and more friendly to the user. By testing the conventional test card and the test card of the Embodiment I by using recombinant protein, it was found that the test card in Embodiment I without the conjugate pad has higher sensitivity than the conventional card strip, and has higher color development at low concentration.

### EMBODIMENT II

A quadruple test card for novel coronavirus/respiratory syncytial virus/influenza A/influenza B comprises a first absorbent pad, a first nitrocellulose membrane, a sample pad, a second nitrocellulose membrane and a second absorbent pad which are sequentially provided on a backing, wherein both ends of the sample pad are respectively lapped to one end of the first nitrocellulose membrane and one end of the second nitrocellulose membrane. The end, away from the sample pad, of the first nitrocellulose membrane is lapped to the first absorbent pad, and the end, away from the sample pad, of the second nitrocellulose membrane is lapped to the second absorbent pad.

A first conjugating zone on the first sample pad is coated with a novel coronavirus antibody labeled with latex beads and a respiratory syncytial virus antibody, and a second conjugating zone on the sample pad is coated with an influenza A antibody labeled with latex beads and an influenza B antibody labeled with latex beads.

The first nitrocellulose membrane is provided with two first test line and a first control line, one of the first test lines is coated with the coronavirus antibody, the other of the first test lines is coated with the respiratory syncytial virus, and the first control line is coated with a goat anti-mouse IgG antibody.

The second nitrocellulose membrane is provided with two test lines and a second control line, one of the second test lines is coated with the influenza A antibody, the other of the second test lines is coated with the influenza B antibody, and the second control line is coated with the goat anti-mouse IgG antibody.

The embodiment II is compared with a conventional test card available on the market, with comparison results as shown in Table 2.

TABLE 2
Testing results of Embodiment II and Conventional Test card

| Comparative | | | | | | | | | |
|---|---|---|---|---|---|---|---|---|---|
| Item | | Embodiment II | | | | Conventional test card | | | |
| Item name | | Novel | Respiratory | Influenza | Influenza | Novel | Respiratory | Influenza | Influenza |
| | | coronavirus | syncytial | A | B | coronavirus | syncytial | A | B |
| | | | virus | | | | virus | | |
| Production | | Excellent | | | | Poor | | | |
| efficiency | | | | | | | | | |
| Sample | | One time (Three drops) | | | | Four times (Twelve drops) | | | |
| injection | | | | | | | | | |
| times | | | | | | | | | |
| Color | | Multi-color | | | | Single color | | | |
| development | | | | | | | | | |
| effect | | | | | | | | | |
| Negative | | − | − | − | − | − | − | − | − |
| matrix | | | | | | | | | |
| 10 | pg/ml | + | + | + | + | ± | ± | ± | ± |
| 20 | pg/ml | ++ | ++ | ++ | ++ | + | + | + | + |
| 40 | pg/ml | ++ | ++ | ++ | ++ | ++ | ++ | ++ | ++ |
| 80 | pg/ml | +++ | +++ | +++ | +++ | +++ | +++ | +++ | +++ |
| 160 | pg/ml | +++ | +++ | +++ | +++ | +++ | +++ | +++ | +++ |

It may be known from the Table 2 that, compared with the conventional test card, the Embodiment II is more productive, less in sample injection times, and more obvious and clearer in color development, and more friendly to the user. By testing the conventional test card and the test card of the Embodiment II by using recombinant protein, it was found that the test card in Embodiment II without the conjugate pad has higher sensitivity than the conventional card strip, and has higher color development at low concentration.

### EMBODIMENT III

A duplex test card for influenza A/novel coronavirus comprises a first absorbent pad, a first nitrocellulose membrane, a sample pad, a second nitrocellulose membrane and a second absorbent pad which are sequentially provided on a backing, wherein both ends of the sample pad are respectively lapped to one end of the first nitrocellulose membrane and one end of the second nitrocellulose membrane. The end, away from the sample pad, of the first nitrocellulose membrane is lapped to the first absorbent pad, and the end, away from the sample pad, of the second nitrocellulose membrane is lapped to the second absorbent pad.

A first conjugating zone on the sample pad is coated with a novel coronavirus antibody labeled with latex beads, and a second conjugating zone on the sample pad is coated with an influenza A antibody labeled with latex beads.

The first nitrocellulose membrane is provided with a first test line and a first control line, the first test line is coated with the novel coronavirus antibody, and the first control line is coated with a goat anti-mouse IgG antibody.

The second nitrocellulose membrane is provided with a second test line and a second control line, the first test line is coated with a goat anti-mouse IgG antibody, and the second test line is coated with the goat anti-mouse IgG antibody.

The embodiment III is compared with a conventional test card available on the market, with comparison results as shown in Table 3:

TABLE 3
Testing results of Embodiment III and Conventional Test card

| Comparative Item | | Embodiment III | | Conventional test card | |
|---|---|---|---|---|---|
| Item name | | Novel | Influenza | Novel | Influenza |
| | | coronavirus | A | coronavirus | A |
| Production | | Excellent | | Poor | |
| efficiency | | | | | |
| Sample injection | | One time (Three drops) | | Three times (Nine drops) | |
| times | | | | | |
| Color development | | Multi-color | | Single color | |
| effect | | | | | |
| Negative matrix | | − | − | − | − |
| 10 | pg/ml | + | + | ± | ± |
| 20 | pg/ml | ++ | ++ | + | + |
| 40 | pg/ml | ++ | ++ | ++ | ++ |
| 80 | pg/ml | +++ | +++ | +++ | +++ |
| 160 | pg/ml | +++ | +++ | +++ | +++ |

It may be known from the Table 3 that, compared with the conventional test card, the Embodiment III is more productive, less in sample injection times, and more obvious and clearer in color development, and more friendly to the user. By testing the conventional test card and the test card of the Embodiment III by using recombinant protein, it was found that the test card in Embodiment III without the conjugate pad has higher sensitivity than the conventional card strip, and has higher color development at low concentration.

It should be noted that the technical solutions of the various embodiments of the present disclosure may be combined with each other, but only on the basis of what those skilled in the art can achieve. When the combination of technical solutions appears to contradict each other or cannot be achieved, it should be considered that such combination of technical solutions does not exist and is not within the scope of protection claimed by the present disclosure.

The above are preferred embodiments of the present disclosure merely and are not intended to limit the patent scope of the present disclosure. Any equivalent structures made according to the description and the accompanying drawings of the present disclosure without departing from the idea of the present disclosure, or any equivalent structures applied in other relevant technical fields directly or indirectly are intended to be comprised in the patent protection scope of the present disclosure.

What is claimed is:

1. A two-dimensional multiplex chromatography test card, said test card comprising a first absorbent pad (11), a first nitrocellulose membrane (10), a sample pad (1), a second nitrocellulose membrane (3) and a second absorbent pad (4) which are sequentially provided on a backing (5) along a length direction of the backing (5);
both ends of the sample pad (1) are respectively lapped to one end of the first nitrocellulose membrane (10) and one end of the second nitrocellulose membrane (3); the end, away from the sample pad, of the first nitrocellulose membrane (10) is lapped to the first absorbent pad (11), and the end, away from the sample pad (1), of the second nitrocellulose membrane (3) is lapped to the second absorbent pad (4);
a first conjugating zone (9) on the sample pad (1) is coated with at least one first latex bead labeled antibody, and a second conjugating zone (2) on the sample pad (1) is coated with at least one second latex bead labeled antibody;
the first nitrocellulose membrane (10) is provided with at least one first test line (12 and/or 13) and a first control line (14), and each of the first test lines is coated with a first antibody corresponding to the first latex bead labeled antibody; and
the second nitrocellulose membrane (3) is provided with at least one second test line (6 and/or 7) and a second control line (8), and each of the second test lines is coated with a second antibody corresponding to the second latex bead labeled antibody.

2. The two-dimensional multiplex chromatography test card according to claim 1, wherein latex beads in each of the first latex bead labeled antibodies and each of the second latex bead labeled antibodies have the same or different colors.

3. The two-dimensional multiplex chromatography test card according to claim 1, wherein the first latex bead labeled antibody comprises an influenza A labeled antibody labeled with latex beads, an influenza B labeled antibody labeled with latex beads, or a novel coronavirus labeled antibody labeled with latex beads; and the second latex bead labeled antibody comprises an influenza A labeled antibody labeled with latex beads, an influenza B labeled antibody labeled with latex beads, or a novel coronavirus labeled antibody labeled with latex beads.

4. The two-dimensional multiplex chromatography test card according to claim 1, wherein each of the first and second control lines are coated with a goat anti-mouse IgG antibody.

5. The two-dimensional multiplex chromatography test card according to claim 1, wherein the sample pad (1) is obtained by infiltrating the sample pad (1) in sample pad treatment buffer and then drying, wherein the sample pad treatment buffer comprises Tris(hydroxymethyl)aminomethane, casein, and Tween-20.

6. The two-dimensional multiplex chromatography test card according to claim 1, wherein the first conjugation zone (9) is 3 mm to 5 mm away from an edge of one end of the sample pad (1), and the second conjugation zone (2) is 3 mm to 5 mm away from an edge of one end of the sample pad (1).

7. The two-dimensional multiplex chromatography test card according to claim 1, wherein the lapping part has a lapping length ranging from 1 mm to 3 mm.

8. The two-dimensional multiplex chromatography test card according to claim 1, wherein the coating amount of each of the first antibodies is 0.1 to 2.0 mg/mL and the coating amount of each of the second antibodies is 0.1 to 2.0 mg/mL.

9. The two-dimensional multiplex chromatography test card according to claim 1, wherein the concentration of the latex beads in each of the first latex bead labeled antibodies is 0.2% to 0.4%, and the concentration of the latex beads in each of the second latex bead labeled antibodies is 0.2% to 0.4%.

10. The two-dimensional multiplex chromatography test card according to claim 1, wherein the latex beads in the first latex bead labeled antibody and in the second latex bead labeled antibody have a particle size of 300 nm to 500 nm.

11. The two-dimensional multiplex chromatography test card according to claim 2, wherein the latex beads in the first latex bead labeled antibody and in the second latex bead labeled antibody have a particle size of 300 nm to 500 nm.

12. The two-dimensional multiplex chromatography test card according to claim 3, wherein the latex beads in the first latex bead labeled antibody and in the second latex bead labeled antibody have a particle size of 300 nm to 500 nm.

13. The two-dimensional multiplex chromatography test card according to claim 4, wherein the latex beads in the first latex bead labeled antibody and in the second latex bead labeled antibody have a particle size of 300 nm to 500 nm.

14. The two-dimensional multiplex chromatography test card according to claim 5, wherein the latex beads in the first latex bead labeled antibody and in the second latex bead labeled antibody have a particle size of 300 nm to 500 nm.

15. The two-dimensional multiplex chromatography test card according to claim 6, wherein the latex beads in the first latex bead labeled antibody and in the second latex bead labeled antibody have a particle size of 300 nm to 500 nm.

16. The two-dimensional multiplex chromatography test card according to claim 7, wherein the latex beads in the first latex bead labeled antibody and in the second latex bead labeled antibody have a particle size of 300 nm to 500 nm.

17. The two-dimensional multiplex chromatography test card according to claim 8, wherein the latex beads in the first latex bead labeled antibody and in the second latex bead labeled antibody have a particle size of 300 nm to 500 nm.

18. The two-dimensional multiplex chromatography test card according to claim 9, wherein the latex beads in the first latex bead labeled antibody and in the second latex bead labeled antibody have a particle size of 300 nm to 500 nm.
